(19) United States
(12) Patent Application Publication
(10) Pub. No.: US 2016/0058293 A1
(43) Pub. Date: Mar. 3, 2016

(54) Photoacoustic Imager

(71) Applicant: Xtrillion, Inc., Tokyo (JP)

(72) Inventor: Yusuke SHIGETA, Tokyo (JP)

(21) Appl. No.: 14/790,547

(22) Filed: Jul. 2, 2015

(30) Foreign Application Priority Data
Aug. 28, 2014 (JP) 2014-173540

(51) Int. Cl. A61B 5/00

(52) U.S. Cl. CPC A61B 5/0095; A61B 5/742

(57) ABSTRACT

This photoacoustic imager includes a detection portion, a light-emitting semiconductor element light source portion arranged in proximity to the detection portion, and a sealing portion configured to propagate an acoustic wave generated by a detection object to the detection portion by sealing a surface of the detection portion on a front side in a detection direction where a specimen is arranged with respect to the detection portion and arranged on the front side in the detection direction where the specimen is arranged with respect to the detection portion.

### BACKGROUND OF THE INVENTION

1. Field of the Invention

The present invention relates to a photoacoustic imager, and more particularly, it relates to a photoacoustic imager including a light source applying light to a specimen and a detection portion detecting an acoustic wave.

2. Description of the Background Art

A photoacoustic imager including a light source applying light to a specimen and a detection portion detecting an acoustic wave is known in general, as disclosed in Japanese Patent Laying-Open No. 2013-75000.

The aforementioned Japanese Patent Laying-Open No. 2013-75000 discloses a photoacoustic imager including a laser beam source applying a laser beam to a specimen and a detection portion detecting an acoustic wave generated by a detection object in a specimen absorbing the laser beam received from the laser beam source. This photoacoustic imager is configured to guide the laser beam from the laser beam source to a probe with an optical fiber member, to apply the laser beam to the specimen and to detect the acoustic wave generated by the specimen in response to the applied laser beam with the detection portion arranged in proximity to the specimen.

However, the photoacoustic imager according to the aforementioned Japanese Patent Laying-Open No. 2013-75000 is disadvantageously increased in size, due to the provision of the laser beam source such as a solid laser. When the laser beam source is replaced with a light-emitting semiconductor element light source employing LED (light-emitting diode) elements or the like in order to miniaturize the photoacoustic imager, however, the light-emitting semiconductor element light source is arranged in proximity to the specimen in order to apply sufficient light (in a quantity allowing detection of the specimen) to the specimen, since the output of the light-emitting semiconductor element light source is small as compared with the output of the laser beam source. When the detection portion and the light-emitting semiconductor element light source are approximated to the specimen in this case, it follows that the detection portion and the light-emitting semiconductor element light source are arranged to be substantially flush with a contact surface of the photoacoustic imager to the specimen. Therefore, light cannot be sufficiently delivered to a shallow portion of the specimen provided immediately under the detection portion and out of an orientation angle of the light from the light-emitting semiconductor element light source outputting diffused light. Consequently, the detection portion cannot conceivably receive a sufficient acoustic wave necessary for detection of the specimen from the shallow portion of the specimen provided immediately under the detection portion.

### SUMMARY OF THE INVENTION

The present invention has been proposed in order to solve the aforementioned problem, and an object of the present invention is to provide a photoacoustic imager capable of receiving a sufficient acoustic wave necessary for detection of a specimen from a shallow portion of the specimen provided immediately under a detection portion.

A photoacoustic imager according to an aspect of the present invention includes a light-emitting semiconductor element light source portion including a light-emitting semiconductor element light source outputting light to be applied to a specimen, a detection portion arranged in proximity to the light-emitting semiconductor element light source portion for detecting an acoustic wave generated by a detection object in the specimen absorbing the light applied to the specimen by the light-emitting semiconductor element light source and a sealing portion configured to propagate the acoustic wave generated by the detection object to the detection portion by sealing a surface of the detection portion on a front side in a detection direction where the specimen is arranged with respect to the detection portion and arranged on the front side in the detection direction where the specimen is arranged with respect to the detection portion.

As hereinabove described, the photoacoustic imager according to the present invention is provided with the sealing portion configured to propagate the acoustic wave generated by the detection object to the detection portion by sealing the surface of the detection portion on the front side in the detection direction and arranged on the front side in the detection direction where the specimen is arranged with respect to the detection portion so that the specimen and the light-emitting semiconductor element light source are arranged to be separated from each other at a prescribed interval due to the sealing portion, whereby the photoacoustic imager can deliver light (diffused light) from the light-emitting semiconductor element light source to a shallow portion of the specimen provided immediately under the detection portion. Therefore, the photoacoustic imager can receive a sufficient acoustic wave necessary for detection of the specimen from the shallow portion of the specimen provided immediately under the detection portion.

In the photoacoustic imager according to the aforementioned aspect, the sealing portion preferably includes a contact surface coming into contact with the specimen arranged on the front side in the detection direction, and is preferably configured to seal not only the surface of the detection portion but also the light-emitting semiconductor element light source in a state in contact with an emitting surface for light emitted from the light-emitting semiconductor element light source of the light-emitting semiconductor element light source portion on the front side in the detection direction and to transmit the light from the light-emitting semiconductor element light source. According to this structure, the photoacoustic imager can apply the light from the light-emitting semiconductor element light source to the specimen through the sealing portion, whereby loss of light can be suppressed as compared with a case of applying the light from the light-emitting semiconductor element light source to the specimen through an air layer. Further, the sealing portion seals the light-emitting semiconductor element light source, not to expose the light-emitting semiconductor element light source. Therefore, the light-emitting semiconductor element light source does not directly come into contact with the specimen, whereby the same can be prevented from wire disconnection resulting from direct contact with the specimen.

In this case, a pair of the light-emitting semiconductor element light source portions are preferably provided to hold the detection portion therebetween and so configured that an intersection of light emitted from the pair of light-emitting semiconductor element light source portions on the side of the detection portion is positioned on the front side of the detection portion in the detection direction, and the distance from the light-emitting semiconductor element light source to the contact surface on the front side in the detection direction is preferably larger than the distance from the light-emitting semiconductor element light source to the intersection on the front side in the detection direction. According to this structure, an intersection (intersection of light on the side of the detection portion) between orientation angles of the light from the pair of light-emitting semiconductor element light sources is arranged inside the sealing portion provided immediately under the detection portion, whereby the photoacoustic imager can deliver light to the whole area of the specimen provided immediately under the detection portion. Consequently, the photoacoustic imager can receive a sufficient acoustic wave necessary for detection of the specimen from the entire shallow portion of the specimen provided immediately under the detection portion.

In the photoacoustic imager according to the aforementioned aspect, the distance from the light-emitting semiconductor element light source to the contact surface on the front side in the detection direction is preferably larger than the thickness of the light-emitting semiconductor element light source. According to this structure, a sufficient interval can be ensured between the light-emitting semiconductor element light source and the contact surface, whereby the photoacoustic imager can deliver more light to the specimen provided immediately under the detection portion.

In the photoacoustic imager according to the aforementioned aspect, the sealing portion preferably covers the detection portion and the light-emitting semiconductor element light source with no clearance. According to this structure, the sealing portion covers the detection portion and the light-emitting semiconductor element light source with no clearance, whereby the detection portion and the light-emitting semiconductor element light source can be prevented from adhesion of water or dust.

In the photoacoustic imager according to the aforementioned aspect, the sealing portion preferably includes a curved surface portion arranged on an end on a front side in an emission direction of the light-emitting semiconductor element light source for sealing the detection portion and the light-emitting semiconductor element light source, and is configured to converge light toward the front side of the detection portion in the detection direction by reflecting and refracting the light from the light-emitting semiconductor element light source on the curved surface portion. According to this structure, the photoacoustic imager can converge the light on the specimen provided immediately under the detection portion by reflecting and refracting the light on the curved surface portion, whereby the shallow portion of the specimen provided immediately under the detection portion can generate a larger quantity of acoustic wave.

In this case, the curved surface portion is preferably provided in the form of an arc smoothly connecting a side end surface of the sealing portion and the contact surface of the sealing portion with each other. According to this structure, the photoacoustic imager can converge the light from the light-emitting semiconductor element light source toward the front side of the detection portion in the detection direction with the simple structure of providing the arcuate shape (R shape) on a corner portion of the sealing portion.

In the aforementioned structure in which the curved surface portion is provided in the form of an arc, the side end surface is preferably configured to be substantially flush with a side end surface of the light-emitting semiconductor element light source opposite to the side of the detection portion. According to this structure, the size of the sealing portion can be reduced as compared with a case where the sealing portion covers the side end surface of the light-emitting semiconductor element light source opposite to the side of the detection portion, whereby the photoacoustic imager can be miniaturized.

The photoacoustic imager according to the aforementioned aspect preferably further includes a cover portion including a contact surface coming into contact with the specimen on the front side in the detection direction and provided in the form of a box opened on the side of the detection portion for propagating the acoustic wave and transmitting the light, and the sealing portion is preferably provided to fill up a space between the cover portion and the detection portion and the light-emitting semiconductor element light source. According to this structure, the sealing portion can fill up (charge) the space between the cover portion and the detection portion and the light-emitting semiconductor element light source, whereby an air layer between the cover portion and the detection portion and the light-emitting semiconductor element light source can be eliminated. Therefore, loss of light can be suppressed as compared with a case of applying light from the light-emitting semiconductor element light source to the specimen through an air layer. Further, the light-emitting semiconductor element light source can be arranged to be further separated from the specimen by the thickness of the cover portion in addition to the thickness of the sealing portion, whereby the photoacoustic imager can deliver the light from the light-emitting semiconductor element light source to a shallower portion of the specimen provided immediately under the detection portion.

In this case, the sealing portion is preferably charged into the cover portion in a state where the detection portion and the light-emitting semiconductor element light source are arranged in the cover portion thereby covering the space between the detection portion and the light-emitting semiconductor element light source and the cover portion with no clearance. According to this structure, the sealing portion can be charged into the cover portion while arranging the detection portion and the light-emitting semiconductor element light source therein, whereby the sealing portion can easily cover the space between the detection portion and the light-emitting semiconductor element light source and the cover portion with no clearance.

In this case, the light-emitting semiconductor element light source preferably has a plurality of light-emitting semiconductor elements and an element sealing portion constituting the light-emitting semiconductor element light source along with the plurality of light-emitting semiconductor elements by sealing the light-emitting semiconductor elements, and the sealing portion preferably has a refractive index larger than the refractive index of the cover portion and not more than the refractive index of the element sealing portion. According to this structure, the photoacoustic imager can refract light from the light-emitting semiconductor element light source toward a portion provided immediately under the detection portion on the boundary surface between the element sealing portion constituting the light-emitting semiconductor element light source and the sealing portion and the boundary surface between the sealing portion and the cover portion, whereby the same can deliver the light to a shallower portion of the specimen provided immediately under the detection portion. In other words, the photoacoustic imager can refract light from the light-emitting semiconductor element light source toward the portion provided immediately under the detection portion with members whose refractive indices are successively reduced, whereby the same can deliver the light to the shallower portion of the specimen provided immediately under the detection portion.

In the photoacoustic imager according to the aforementioned aspect, the light-emitting semiconductor element light source preferably has a plurality of light-emitting semiconductor elements and an element sealing portion constituting the light-emitting semiconductor element light source along with the plurality of light-emitting semiconductor elements by sealing the light-emitting semiconductor elements, and the sealing portion preferably has a refractive index larger than the refractive index of the specimen and not more than the refractive index of the element sealing portion. According to this structure, the photoacoustic imager can refract light from the light-emitting semiconductor element light source toward a portion provided immediately under the detection portion on the boundary surface between the element sealing portion constituting the light-emitting semiconductor element light source and the sealing portion and the boundary surface between the sealing portion and the specimen, whereby the same can deliver the light to a shallower portion of the specimen provided immediately under the detection portion. In other words, the photoacoustic imager can refract the light from the light-emitting semiconductor element light source toward the portion provided immediately under the detection portion with members whose refractive indices are successively reduced, whereby the same can deliver the light to the shallower portion of the specimen provided immediately under the detection portion as compared with a case of directly delivering the light without refraction.

In the photoacoustic imager according to the aforementioned aspect, the detection portion preferably includes an ultrasonic vibrator detecting the acoustic wave as an ultrasonic wave and an acoustic lens arranged on the front side of the ultrasonic vibrator in the detection direction in a state sealed by the sealing portion for converging the acoustic wave from the detection object on the ultrasonic vibrator, and the sealing portion preferably has larger transmittance than the acoustic lens and equivalent ultrasonic wave propagation loss to the acoustic lens. According to this structure, the photoacoustic imager can efficiently propagate the acoustic wave to the ultrasonic vibrator by converging the same with the acoustic lens. Further, the sealing portion has the larger light transmittance than the acoustic lens and the equivalent ultrasonic wave propagation loss to the acoustic lens, whereby the photoacoustic imager can apply light to the specimen with small loss and also propagate the acoustic wave to the detection portion with small loss.

In this case, the acoustic lens is preferably provided in a rounded convex shape protruding on the front side in the detection direction. According to this structure, the acoustic lens can refract the acoustic wave toward the ultrasonic vibrator due to the rounded convex shape protruding on the front side in the detection direction, thereby efficiently converging the acoustic wave on the ultrasonic vibrator.

In the aforementioned structure in which the detection portion includes the ultrasonic vibrator and the acoustic lens, the sealing portion is preferably arranged between the acoustic lens and the light-emitting semiconductor element light source with no clearance. According to this structure, the photoacoustic imager can transmit the light and propagate the acoustic wave also between the acoustic lens and the light-emitting semiconductor element light source. Consequently, the photoacoustic imager can suppress energy loss between the acoustic wave and the light-emitting semiconductor element light source.

In the aforementioned structure in which the detection portion includes the ultrasonic vibrator and the acoustic lens, the sealing portion preferably includes a contact surface coming into contact with the specimen arranged on the front side in the detection direction, and the contact surface is preferably arranged substantially parallelly with a light-emitting surface of the light-emitting semiconductor element light source on the front side in the detection direction and a surface of the ultrasonic vibrator on the front side in the detection direction. According to this structure, the contact surface is so parallelly arranged with the light-emitting surface and the surface of the ultrasonic vibrator on the front side in the detection direction that the photoacoustic imager can efficiently deliver the light from the light-emitting surface to the contact surface (the specimen) and can also efficiently deliver the acoustic wave from the contact surface to the detection portion.

In the photoacoustic imager according to the aforementioned aspect, the detection portion and the light-emitting semiconductor element light source are preferably formed to extend in the same direction orthogonal to the front side in the detection direction. According to this structure, the detection portion can detect the acoustic wave from a wider range in a case where the detection portion and the light-emitting semiconductor element light source are provided in slender shapes while keeping the positional relation therebetween.

In the photoacoustic imager according to the aforementioned aspect, the light-emitting semiconductor element light source portion preferably includes a light-emitting diode element as a light-emitting semiconductor element. According to this structure, the photoacoustic imager can detect the detection object by employing the light-emitting diode element requiring relatively small power consumption.

In the photoacoustic imager according to the aforementioned aspect, the light-emitting semiconductor element light source portion preferably includes a semiconductor laser element as a light-emitting semiconductor element. According to this structure, the photoacoustic imager can apply a laser beam relatively higher in directivity as compared with a light-emitting diode element to the specimen, whereby the same can reliably apply most part of the light from the semiconductor laser element to the specimen.

In the photoacoustic imager according to the aforementioned aspect, the light-emitting semiconductor element light source portion preferably includes an organic light-emitting diode element as a light-emitting semiconductor element. According to this structure, the light-emitting semiconductor element light source portion can be easily miniaturized due to the employment of the organic light-emitting diode element easily reducible in thickness.

According to the present invention, as hereinabove described, a photoacoustic imager capable of receiving a sufficient acoustic wave necessary for detection of a specimen from a shallow portion of the specimen provided immediately under a detection portion can be provided.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a perspective view showing the overall structure of a photoacoustic imager according to each of first to third embodiments of the present invention;

FIG. 2 is a block diagram showing the overall structure of the photoacoustic imager according to each of the first to third embodiments of the present invention;

FIG. 3 illustrates a specimen arranged with respect to a sectional view taken along the line 900-900 in FIG. 1;

FIG. 4 is an enlarged sectional view showing a detection portion, LED light source portions and a sealing portion of the photoacoustic imager according to the second embodiment of the present invention;

FIG. 5 is an enlarged sectional view showing a detection portion, LED light source portions and a sealing portion of the photoacoustic imager according to the third embodiment of the present invention;

FIG. 6 is an enlarged sectional view showing a detection portion, LED light source portions and a sealing portion of a photoacoustic imager according to a fourth embodiment of the present invention;

FIG. 7 is a diagram for illustrating refraction of light emitted from LED elements of a photoacoustic imager according to a first modification of each of the first to fourth embodiments of the present invention;

FIG. 8 illustrates light-emitting semiconductor elements of a photoacoustic imager according to a second modification of each of the first to fourth embodiments of the present invention; and

FIG. 9 is a perspective view showing the overall structure of a photoacoustic imager according to a third modification of each of the first to fourth embodiments of the present invention.

### DESCRIPTION OF THE PREFERRED EMBODIMENTS

Embodiments of the present invention are now described with reference to the drawings.

### First Embodiment

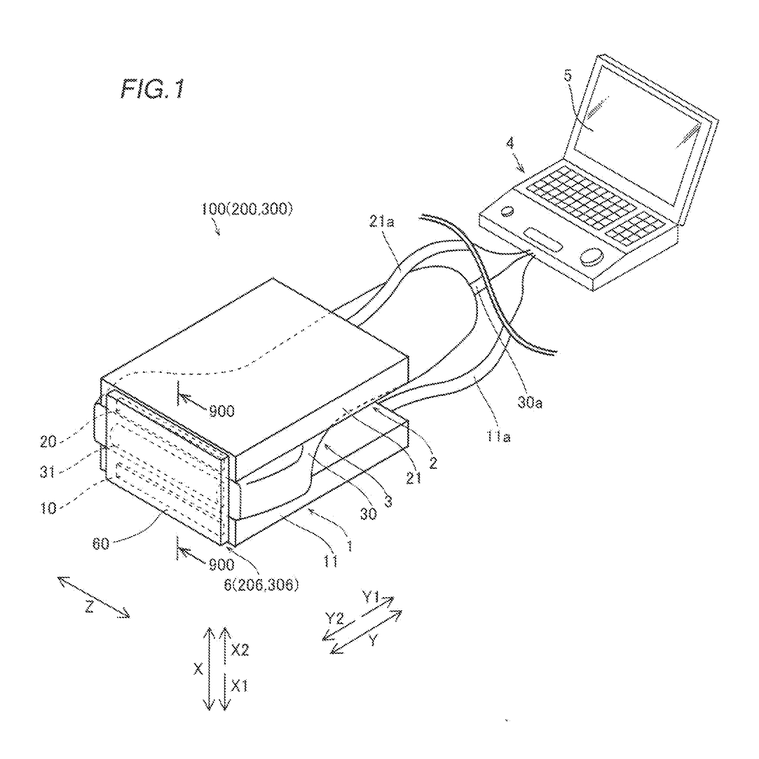

First, the structure of a photoacoustic imager 100 according to a first embodiment of the present invention is described with reference to FIGS. 1 to 3.

The photoacoustic imager 100 according to the first embodiment of the present invention includes two LED (light-emitting diode) light source portions 1 and 2 including LED light sources 10 and 20 respectively, a detection portion 3, a signal processing portion 4, a display portion 5 and a sealing portion 6, as shown in FIG. 1. The LED light sources 10 and 20 are both examples of the “light-emitting semiconductor element light source” in the present invention. The LED light source portions 1 and 2 are both examples of the “light-emitting semiconductor element light source portion” in the present invention.

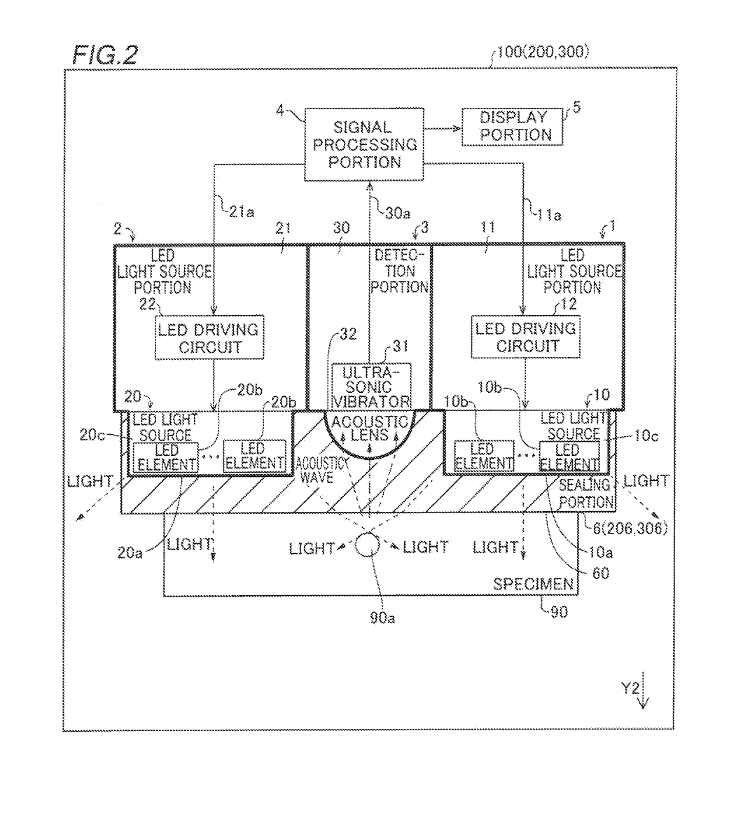

As shown in FIG. 2, the photoacoustic imager 100 is configured to apply light to a specimen 90 such as a human body from the LED light source portions 1 and 2. Further, the photoacoustic imager 100 is configured to detect an acoustic wave generated by a detection object 90a in the specimen 90 absorbing the applied light with the detection portion 3. In addition, the photoacoustic imager 100 is configured to be capable of specifying and imaging the detection object 90a on the basis of the acoustic wave detected by the detection portion 3.

According to the first embodiment, the photoacoustic imager 100 is so configured that the sealing portion 6 seals a surface of the detection portion 3 on a front side (along arrow Y2) in a detection direction. Thus, the photoacoustic imager 100 is configured to propagate the acoustic wave generated by the detection object 90a to the detection portion 3 with the sealing portion 6.

Further, the photoacoustic imager 100 is so configured that the sealing portion 6 seals not only the surface of the detection portion 3 on the front side (along arrow Y2) in the detection direction but also the LED light sources 10 and 20 in a state where emitting surfaces 10a and 20a for light emitted the LED light sources 10 and 20 of the LED light source portions 1 and 2 on the front side (along arrow Y2) in the detection direction and the sealing portion 6 are in contact with each other. Details of the sealing portion 6 are described later.

The structures of respective portions of the photoacoustic imager 100 are now described.

As shown in FIG. 2, the LED light source portions 1 and 2 further include housing portions 11 and 21 and LED driving circuits 12 and 22 respectively.

The housing portions 11 and 21 of the LED light source portions 1 and 2 store the LED driving circuits 12 and 22 respectively, and are mounted with the LED light sources 10 and 20 on the front side (along arrow Y2) in the detection direction. Further, the housing portions 11 and 21 are connected with the signal processing portion 4 through cables 11a and 21a respectively.

The LED driving circuits 12 and 22 are configured to control current flowing in the corresponding LED light sources 10 and 20 respectively on the basis of control signals received from the signal processing portion 4. More specifically, the LED driving circuits 12 and 22 are configured to on-off control the current flowing in the corresponding LED light sources 10 and 20 and to control magnitudes (current values) of the current on the basis of the control signals received from the signal processing portion 4.

The LED light sources 10 and 20 have a plurality of LED elements 10b, a plurality of LED elements 20b and element sealing portions 10c and 20c respectively. The LED elements 10b and 20b are both examples of the “light-emitting semiconductor element” in the present invention.

The element sealing portions 10c and 20c constitute the LED light sources 10 and 20 by sealing the plurality of LED elements 10b and the plurality of LED elements 20b respectively. The element sealing portions 10c and 20c are made of silicon-based resin, for example.

The LED light sources 10 and 20 are configured to emit light of substantially identical wavelengths (light having wavelengths of about 700 nm to about 1000 nm, for example). More detailedly, the LED light sources 10 and 20 are configured to emit light corresponding to current fed to the LED elements 10b and 20b on the basis of current control by the LED driving circuits 12 and 22.

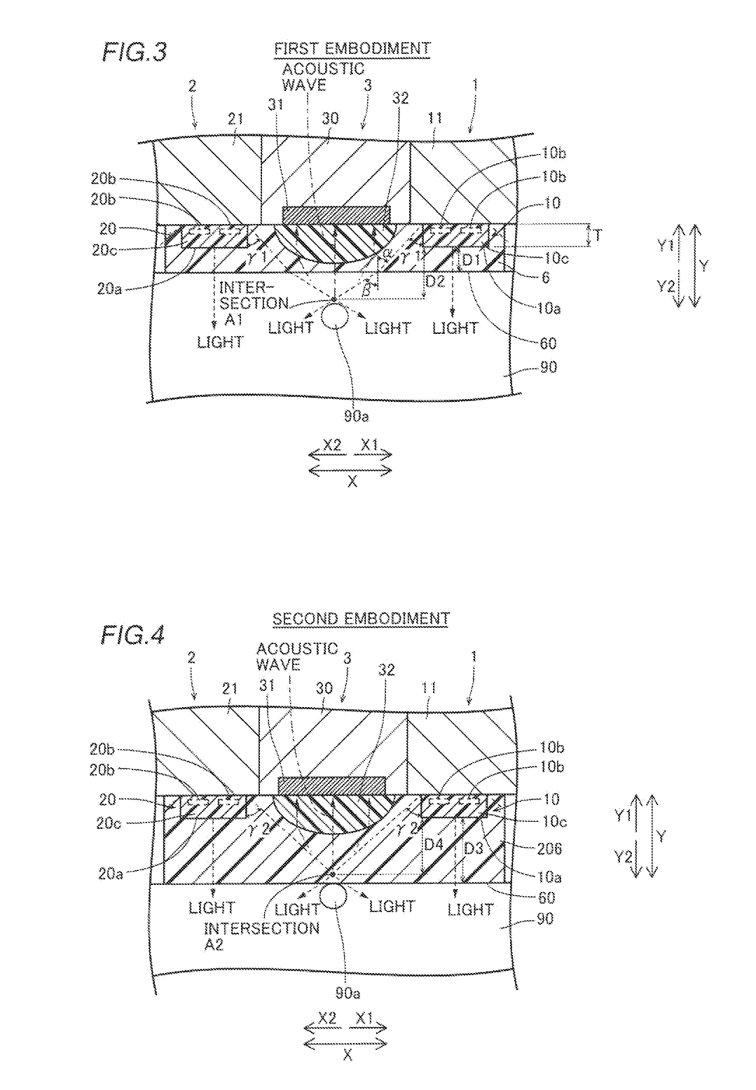

As shown in FIG. 3, the LED light source portions 1 and 2 are provided in a pair, to hold the detection portion 3 therebetween. Further, the LED light source portions 1 and 2 are arranged to extend in a prescribed direction (a direction Z, see FIG. 1) as a whole. In addition, the pair of LED light source portions 1 and 2 are so configured that an intersection A1 between orientation angles γ1 of the emitted light is positioned on the front side (along arrow Y2) of the detection portion 3 in the detection direction. Each orientation angle γ1 indicates an angular range in which the LED light source 10 (20) can output light with respect to a front side (along arrow Y2) in a light emission direction of the LED light source 10 (20).

The LED light sources 10 and 20 are arranged in proximity to an ultrasonic vibrator 31 described later. More detailedly, the LED light sources 10 and 20 are arranged in proximity to a first side (along arrow X1) and a second side (along arrow X2) of the ultrasonic vibrator (the detection portion 3) respectively. Therefore, the LED light sources 10 and 20 are configured to be capable of applying light to the specimen 90 (the detection object 90a) from positions different from each other. The LED light source 10 (20) and the detection portion 3 are arranged on substantially identical planes parallel to a contact surface 60, described later, of the sealing portion 6 in contact with the specimen 90.

The detection portion 3 includes a housing portion 30, the ultrasonic vibrator 31 and an acoustic lens 32.

As shown in FIG. 2, the detection portion 3 is configured to detect an acoustic wave (an ultrasonic wave) vibrating the ultrasonic vibrator 31. The detection portion 3 is further configured to output a signal corresponding to the detected acoustic wave to the signal processing portion 4. The ultrasonic vibrator 31 is arranged to extend in a prescribed direction (a direction Z) as a whole.

The housing portion 30 of the detection portion 3 is mounted with the ultrasonic vibrator 31 and the acoustic lens 32 on the front side (along arrow Y2) in the detection direction. The housing portion 30 is connected with the signal processing portion 4 through a cable 30a.

The acoustic lens 32 is arranged on the front side (along arrow Y2) of the ultrasonic vibrator 31 in the detection direction in a state in contact with the ultrasonic vibrator 31. The acoustic lens 32 is configured to converge an acoustic wave from the detection object 30a on the ultrasonic vibrator 31. More detailedly, the acoustic lens 32 is provided in a rounded convex shape protruding on the front side (along arrow Y2) in the detection direction in side elevational view (as viewed from the direction Z). The acoustic lens 32 is further configured to refract the acoustic wave from the detection object 90a due to the rounded convex shape and to converge the refracted acoustic wave on the ultrasonic vibrator 31.

The signal processing portion 4 is configured to perform imaging by processing a signal detected by the ultrasonic vibrator 31. More detailedly, the signal processing portion 4 includes a CPU (not shown) and a storage portion (not shown) such as a ROM or a RAM, and is configured to process a signal corresponding to the acoustic wave detected by the detection portion 3. The signal processing portion 4 is configured to specify and image the detection object 90a on the basis of the signal corresponding to the acoustic wave generated by the detection object 90a in the specimen 90 and detected by the detection portion 3 in a case of measuring the specimen 90, for example. The signal processing portion 4 is further configured to control the display portion 5 to display an image of the detection object 90a formed in this manner.

The display portion 5 is configured to be capable of displaying the image of the detection object 90a in the specimen 90 and various screens (an operation screen, an information screen and the like) on the basis of control by the signal processing portion 4.

As shown in FIG. 3, the sealing portion 6 is arranged on the front side (along arrow Y2) in the detection direction where the specimen 90 is arranged with respect to the detection portion 3. Further, the sealing portion 6 is arranged on the front side (along arrow Y2) in the light emission direction where the specimen 90 is arranged with respect to the LED light sources 10 and 20. In addition, the sealing portion 6 includes the contact surface 60 in contact with the specimen 90 on the front side (along arrow Y2) in the detection direction. The sealing portion 6 is made of the same silicon-based resin as the element sealing portions 10c and 20c of the LED light sources 10 and 20, for example. Therefore, the sealing portion 6 has the same refractive index as the element sealing portions 10c and 20c of the LED light sources 10 and 20.

The sealing portion 6 is configured to seal the surface of the detection portion 3 on the front side (along arrow Y2) in the detection direction, as described above. More detailedly, the sealing portion 6 seals the acoustic lens 32 provided on the front side (along arrow Y2) of the detection portion 3 in the detection direction and substantially the whole area of the surface of the housing portion 30 along arrow Y2. Therefore, the sealing portion 6 is configured to be capable of propagating the acoustic wave between the specimen 90 (the contact surface 60) and the detection portion 3. The sealing portion 6 covers the detection portion 3 and the LED light source 10 (20) with no clearance. Further, the sealing portion 6 is arranged between the acoustic lens 32 and the LED light source 10 (20) with no clearance. The contact surface 60 is arranged substantially parallelly with the emitting surface 10a (20a) for the light from the LED light source (20) and the surface of the ultrasonic vibrator 31 on the front side (along arrow Y2) in the detection direction.

The sealing portion 6 is configured to seal the LED light sources 10 and 20 in a state in contact with the emitting surfaces 10a and 20a, as hereinabove described. More detailedly, the sealing portion 6 seals the whole LED light sources 10 and 20 and substantially the whole areas of the surfaces of the housing portions 11 and 21 along arrow Y2. Therefore, the sealing portion 6 is configured to be capable of propagating light between the LED light sources 10 and 20 and the specimen 90 (the contact surface 60). Thus, the sealing portion 6 is so provided as to cover and seal regions (the surfaces of the housing portions 11, 21 and 30 along arrow Y2) including the LED light sources 10 and 20 and the acoustic lens 32 with no clearance. Further, the sealing portion 6 has larger light transmittance than the acoustic lens 32 and equivalent ultrasonic wave propagation loss to the acoustic lens 32.

In addition, the sealing portion 6 has a larger refractive index than the specimen 90 (an organism). Assuming that α represents an incidence angle of light from the sealing portion 6 into the specimen 90 on the boundary surface (the contact surface 60) between the sealing portion 6 and the specimen 90 and β represents a refraction angle, the refraction angle β is larger than the incidence angle α.

The sealing portion 6 is so configured that the distance D1 from the LED light source 10 (20) to the contact surface 60 on the front side (along arrow Y2) in the detection direction is smaller than the distance D2 from the LED light source 10 (20) to the intersection A1 on the front side (along arrow Y2) in the detection direction. In other words, the intersection A1 between the orientation angles γ1 of the light from the pair of LED light sources 10 and 20 is arranged inside the specimen 90. An end of the sealing portion 6 on the front side (along arrow Y2) in the light emission direction of the LED light source 10 (20) is angularly formed. The distance D1 is larger than the thickness T of the LED light source 10 (20).

According to the first embodiment, the following effects can be attained:

According to the first embodiment, as hereinabove described, the photoacoustic imager 100 is provided with the sealing portion 6 configured to propagate the acoustic wave generated by the detection object 90a to the detection portion 3 by sealing the surface of the detection portion 3 on the front side in the detection direction and arranged on the front side (along arrow Y2) in the detection direction where the specimen 90 is arranged with respect to the detection portion 3 so that the specimen 90 and the LED light source 10 (20) are arranged to be separated from each other at a prescribed interval due to the sealing portion 6, whereby the photoacoustic imager 100 can deliver the light (diffused light) from the LED light source 10 (20) to a shallow portion of the specimen 90 provided immediately under the detection portion 3. Therefore, the photoacoustic imager 100 can receive a sufficient acoustic wave necessary for detection of the specimen 90 from the shallow portion of the specimen 90 provided immediately under the detection portion 3.

According to the first embodiment, as hereinabove described, the sealing portion 6 is configured to include the contact surface 60 in contact with the specimen 90 arranged on the front side in the detection direction, to seal not only the surface of the detection portion 3 but also the LED light source 10 (20) in the state in contact with the emitting surface 10a (20a) for the light emitted from the LED light source 10 (20) of the LED light source portion 1 (2) on the front side in the detection direction and to transmit the light from the LED light source 10 (20). Thus, the photoacoustic imager 100 can apply the light from the LED light source 10 (20) to the specimen 90 through the sealing portion 6, whereby loss of light can be suppressed as compared with a case of applying the light from the LED light source 10 (20) to the specimen 90 through an air layer. Further, the sealing portion 6 seals the LED light source 10 (20), not to expose the LED light source 10 (20). Therefore, the LED light source 10 (20) does not directly come into contact with the specimen 90, whereby the LED light source 10 (20) can be prevented from wire disconnection resulting from direct contact with the specimen 90.

According to the first embodiment, as hereinabove described, the distance D1 from the LED light source 10 (20) to the contact surface 60 on the front side in the detection direction is rendered larger than the thickness T of the LED light source 10 (20). Thus, a sufficient interval can be ensured between the LED light source 10 (20) and the contact surface 60, whereby the photoacoustic imager 100 can deliver more light to the specimen 90 provided immediately under the detection portion 3.

According to the first embodiment, as hereinabove described, the sealing portion 6 covers the detection portion 3 and the LED light source 10 (20) with no clearance. Thus, the detection portion 3 and the LED light source 10 (20) are so covered with the sealing portion 6 with no clearance that the same can be prevented from adhesion of water or dust.

According to the first embodiment, as hereinabove described, the LED light source 10 (20) is provided with the plurality of LED elements 10b (20b) and the element sealing portion 10c (20c) constituting the LED light source 10 (20) along with the plurality of LED elements 10b (20b) by sealing the LED elements 10b (20b), and the sealing portion 6 is configured to have the refractive index larger than that of the specimen 90 and not more than that of the element sealing portion 10c (20c). Thus, the photoacoustic imager 100 can refract light from the LED light source 10 (20) toward a portion provided immediately under the detection portion 3 on the boundary surface between the element sealing portion 10c (20c) constituting the LED light source 10 (20) and the sealing portion 6 and the boundary surface between the sealing portion 6 and the specimen 90, whereby the same can deliver the light to a shallower portion of the specimen 90 provided immediately under the detection portion 3. In other words, the photoacoustic imager 100 can refract the light from the LED light source 10 (20) (the LED elements 10b (20b)) toward the portion provided immediately under the detection portion 3 with members whose refractive indices are successively reduced, whereby the same can deliver the light to the shallower portion of the specimen 90 provided immediately under the detection portion 3 as compared with a case of directly delivering the light without refraction.

According to the first embodiment, as hereinabove described, the detection portion 3 is provided with the ultrasonic vibrator 31 detecting the acoustic wave as an ultrasonic wave and the acoustic lens 32 arranged on the front side of the ultrasonic vibrator 31 in the detection direction in the state sealed with the sealing portion 6 for converging the acoustic wave from the detection object 90a on the ultrasonic vibrator 31, and the sealing portion 6 is configured to have the larger light transmittance than the acoustic lens 32 and the equivalent ultrasonic wave propagation loss to the acoustic lens 32. Thus, the photoacoustic imager 100 can efficiently propagate the acoustic wave to the ultrasonic vibrator 31 by converging the same with the acoustic lens 32. Further, the sealing portion 6 has the larger light transmittance than the acoustic lens 32 and the equivalent ultrasonic wave propagation loss to the acoustic lens 32, whereby the photoacoustic imager 100 can apply the light to the specimen 90 with small loss and also propagate the acoustic wave to the detection portion 3 with small loss.

According to the first embodiment, as hereinabove described, the acoustic lens 32 is provided in the rounded convex shape protruding on the front side in the detection direction. Thus, the acoustic lens 32 can refract the acoustic wave toward the ultrasonic vibrator 31 due to the rounded convex shape protruding on the front side in the detection direction, thereby efficiently converging the acoustic wave on the ultrasonic vibrator 31.

According to the first embodiment, as hereinabove described, the sealing portion 6 is arranged between the acoustic lens 32 and the LED light source 10 (20) with no clearance. Thus, the photoacoustic imager 100 can transmit the light and propagate the acoustic wave also between the acoustic lens 32 and the LED light source 10 (20). Consequently, the photoacoustic imager 100 can suppress energy loss between the acoustic lens 32 and the LED light source 10 (20).

According to the first embodiment, as hereinabove described, the sealing portion 6 is provided with the contact surface 60 in contact with the specimen 90 arranged on the front side in the detection direction, and the contact surface 60 is arranged substantially parallelly with the emitting surface 10a (20a) for the light from the LED light source 10 (20) on the front side in the detection direction and the surface of the ultrasonic vibrator 31 on the front side in the detection direction. Thus, the contact surface 60 is so arranged substantially parallelly with the emitting surface 10a (20a) and the surface of the ultrasonic vibrator 31 on the front side in the detection direction that the photoacoustic imager 100 can efficiently deliver the light from the emitting surface 10a (20a) to the contact surface (the specimen 90, and can also efficiently deliver the acoustic wave from the contact surface 60 to the detection portion 3.

According to the first embodiment, as hereinabove described, the detection portion 3 and the LED light source 10 (20) are formed to extend in the same direction orthogonal to the front side in the detection direction. Thus, the detection portion 3 can detect the acoustic wave from a wider range in a case where the detection portion 3 and the LED light source 10 (20) are provided in slender shapes while keeping the positional relation therebetween.

According to the first embodiment, as hereinabove described, the LED light source 10 (20) is provided with the LED elements 10b (20b) as light-emitting semiconductor elements. Thus, the photoacoustic imager 100 can detect the detection object 90a by employing the LED elements 10b (20b) requiring relatively small power consumption.

### Second Embodiment

A second embodiment of the present invention is now described with reference to FIGS. 1, 2 and 4. According to the second embodiment, an intersection A2 between orientation angles γ2 of light from a pair of LED light sources 10 and 20 is arranged inside a sealing portion 206, dissimilarly to the aforementioned first embodiment in which the intersection A1 between the orientation angles γ1 of the light from the pair of LED light sources 10 and 20 is arranged inside the specimen 90. Structures of the second embodiment similar to those of the aforementioned first embodiment are denoted by the same reference signs as those in the first embodiment, and redundant description is omitted.

In a photoacoustic imager 200 (see FIGS. 1 and 2) according to the second embodiment, a pair of LED light source portions 1 and 2 are provided to hold a detection portion 3 therebetween, and so configured that the intersection A2 between the orientation angles γ2 of light emitted from the pair of LED light source portions 1 and 2 is positioned on a front side (along arrow Y2) of the detection portion 3 in a detection direction, as shown in FIG. 4.

The sealing portion 206 is so configured that the distance D3 from the LED light source 10 (20) to a contact surface 60 on the front side (along arrow Y2) in the detection direction is larger than the distance D4 from the LED light source 10 (20) to the intersection A2 on the front side (along arrow Y2) in the detection direction. In other words, the intersection A2 between the orientation angles γ2 of the light from the pair of LED light sources 10 and 20 is arranged inside the sealing portion 206.

According to the second embodiment, the following effects can be attained:

According to the second embodiment, the photoacoustic imager 200 is provided with the sealing portion 206 configured to propagate an acoustic wave generated by a detection object 90a to the detection portion 3 by sealing a surface of the detection portion 3 on the front side in the detection direction and arranged on the front side in the detection direction where a specimen 90 is arranged with respect to the detection portion 3, to be capable of receiving a sufficient acoustic wave necessary for detection of the specimen 90 from a shallow portion of the specimen 90 provided immediately under the detection portion 3, similarly to the aforementioned first embodiment.

According to the second embodiment, as hereinabove described, the pair of LED light sources 1 and 2 are provided to hold the detection portion 3 therebetween and so configured that the intersection A2 (intersection of light on the side of the detection portion 3) between the orientation angles γ2 of the light emitted from the pair of LED light source portions 1 and 2 is positioned on the front side of the detection portion 3 in the detection direction, and the distance D3 from the LED light source (20) to the contact surface 60 on the front side in the detection direction is rendered larger than the distance D4 from the LED light source 10 (20) to the intersection A2 on the front side in the detection direction. Thus, the intersection A2 between the orientation angles γ2 of the light from the pair of LED light sources 10 and 20 is arranged inside the sealing portion 206 provided immediately under the detection portion 3, whereby the photoacoustic imager 200 can deliver the light to the whole area of the specimen 90 provided immediately under the detection portion 3. Consequently, the photoacoustic imager 200 can receive a sufficient acoustic wave necessary for detection of the specimen 90 from the entire shallow portion of the specimen 90 provided immediately under the detection portion 3.

### Third Embodiment

A third embodiment of the present invention is now described with reference to FIGS. 1, 2 and 5. According to the third embodiment, an end of a sealing portion 306 on a front side in a light emission direction of an LED light source 10 (20) is provided in the form of a curved surface, dissimilarly to the aforementioned first embodiment in which the end of the sealing portion 6 on the front side in the light emission direction of the LED light source 10 (20) is angularly formed. Structures of the third embodiment similar to those of the aforementioned first embodiment are denoted by the same reference signs as those in the first embodiment, and redundant description is omitted.

In a photoacoustic imager 300 (see FIGS. 1 and 2) according to the third embodiment, the sealing portion 306 includes curved surface portions 306a sealing a detection portion 3 and LED light sources 10 and 20 and arranged on ends on the front side (along arrow Y2) in the light emission direction of the LED light sources 10 and 20 respectively, as shown in FIG. 5. More detailedly, the sealing portion 306 includes the curved surface portions 306a on the respective ends on the front side (along arrow Y2) in the light emission direction of the LED light sources 10 and 20 and respective ends of a contact surface 60 along arrows X1 and X2. The curved surface portions 306a are provided in the form of arcs smoothly connecting side end surfaces 306b (along arrows X1 and X2) of the sealing portion 306 and the contact surface 60 thereof with each other. The side end surfaces 306b of the sealing portion 306 are configured to be substantially flush with a side end surface 10d (20d) of the LED light source 10 (20). The sealing portion 306 is configured to converge light toward a front side (along arrow Y2) of the detection portion 3 in a detection direction by reflecting and refracting the light from the LED light sources 10 and 20 on the curved surface portions 306a.

Further, the sealing portion 306 is so configured that an end surface thereof along arrow X1 is flush with an end surface of the LED light source 10 along arrow X1 in side elevational view (as viewed from a direction Z). In addition, the sealing portion 306 is so configured that an end surface thereof along arrow X2 is flush with an end surface of the LED light source 20 along arrow X2 in side elevational view (as viewed from the direction Z).

According to the third embodiment, the following effects can be attained:

According to the third embodiment, the photoacoustic imager 300 is provided with the sealing portion 306 configured to propagate an acoustic wave generated by a detection object 90a to the detection portion 3 by sealing a surface of the detection portion 3 on the front side in the detection direction and arranged on the front side in the detection direction where a specimen 90 is arranged with respect to the detection portion 3, to be capable of receiving a sufficient acoustic wave necessary for detection of the specimen 90 from a shallow portion of the specimen 90 provided immediately under the detection portion 3, similarly to the aforementioned first embodiment.

According to the third embodiment, as hereinabove described, the sealing portion 306 is provided with the curved surface portions 306a sealing the detection portion 3 and the LED light sources 10 and 20 and arranged on the ends on the front side in the light emission direction of the LED light sources 10 and 20, and configured to converge light toward the front side of the detection portion 3 in the detection direction by reflecting and refracting the light from the LED light sources 10 and 20 on the curved surface portions 306a. Thus, the photoacoustic imager 300 can converge the light on the specimen 90 provided immediately under the detection portion 3 by reflecting and refracting the light on the curved surface portions 306a, whereby the shallow portion of the specimen 90 provided immediately under the detection portion 3 can generate a larger quantity of acoustic wave.

According to the third embodiment, as hereinabove described, the curved surface portions 306a are provided in the form of arcs smoothly connecting the side end surfaces 306b and the contact surface 60 of the sealing portion 306 with each other. Thus, the photoacoustic imager 300 can converge the light from the LED light source 10 (20) toward the front side of the detection portion 3 in the detection direction with the simple structure of providing the arcuate shapes (R shapes) on corner portions of the sealing portion 306.

According to the third embodiment, as hereinabove described, the photoacoustic imager 300 is so configured that the side end surfaces 306b of the sealing portion 306 are substantially flush with the side end surface 10d (20d) of the LED light source 10 (20) opposite to the side of the detection portion 3. Thus, the size of the sealing portion 306 can be reduced as compared with a case where the sealing portion 306 covers the side end surface 10d (20d) of the LED light source 10 (20) opposite to the side of the detection portion 3, whereby the photoacoustic imager 300 can be miniaturized.

### Fourth Embodiment

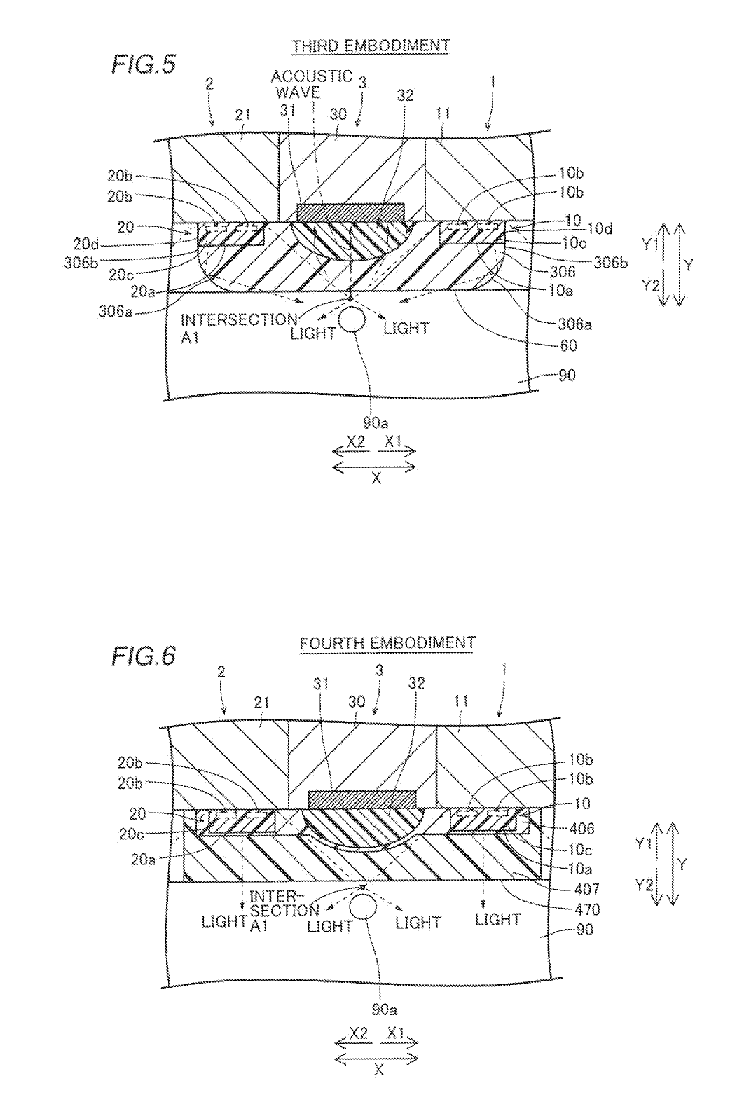

A fourth embodiment of the present invention is now described with reference to FIG. 6. According to the fourth embodiment, a cover portion 407 filling up (charging) a sealing portion 406 is configured to include a contact surface 470 coming into contact with a specimen 90, dissimilarly to the aforementioned first embodiment in which the sealing portion 6 is configured to include the contact surface 60 coming into contact with the specimen 90. Structures of the fourth embodiment similar to those of the aforementioned first embodiment are denoted by the same reference signs as those in the first embodiment, and redundant description is omitted.

As shown in FIG. 6, a photoacoustic imager (not shown) according to the fourth embodiment includes the cover portion 407 including the contact surface 470 coming into contact with the specimen 90 on a front side (along arrow Y2) in a detection direction and provided in the form of a box opened on the side (along arrow Y1) of a detection portion 3.

More detailedly, the cover portion 407 is provided in the form of a box having an opening on the side along arrow Y1. Further, the cover portion 407 includes the contact surface 470 coming into contact with the specimen 90 on the front side (along arrow Y2) of a bottom portion, opposite to the opening, in the detection direction. The cover portion 407 is made of acrylic resin, for example.

The sealing portion 406 is provided to fill up a space between the cover portion 407 and the detection portion 3 and an LED light source 10 (20).

More detailedly, the sealing portion 406 is formed by being charged into the space between the cover portion 407 and the detection portion 3 and the LED light source 10 (20) in a state where the cover portion 407 (the contact surface 470) is arranged on the front side (along arrow Y2) (20) in the detection direction with respect to the detection portion 3 and the LED light source 10.

The sealing portion 406 is charged into the cover portion 407 in a state where the detection portion 3 and the LED light source 10 (20) are arranged in the cover portion 407, thereby covering the space between the cover portion 407 and the detection portion 3 and the LED light source 10 (20) with no clearance. Further, the sealing portion 406 is charged into the space between the cover portion 407 and the detection portion 3 and the LED light source 10 (20), thereby integrally fixing the detection portion 3 and the LED light source 10 (20) to each other.

The sealing portion 406 has a larger refractive index than the cover portion 407.

According to the fourth embodiment, the following effects can be attained:

According to the fourth embodiment, the photoacoustic imager is provided with the sealing portion 406 configured to propagate an acoustic wave generated by a detection object 90a to the detection portion 3 by sealing a surface of the detection portion 3 on the front side in the detection direction and arranged on the front side in the detection direction where a specimen 90 is arranged with respect to the detection portion 3, to be capable of receiving a sufficient acoustic wave necessary for detection of the specimen 90 from a shallow portion of the specimen 90 provided immediately under the detection portion 3, similarly to the aforementioned first embodiment.

According to the fourth embodiment, as hereinabove described, the photoacoustic imager is provided with the cover portion 407 including the contact surface 60 coming into contact with the specimen 90 on the front side in the detection portion and provided in the form of a box opened on the side of the detection portion 3 for propagating the acoustic wave and transmitting light, and the sealing portion 406 is provided to fill up the space between the cover portion 407 and the detection portion 3 and the LED light source 10 (20). Thus, the sealing portion 406 can fill up (charge) the space between the cover portion 407 and the detection portion 3 and the LED light source 10 (20), whereby an air layer between the cover portion 407 and the detection portion 3 and the LED light source 10 (20) can be eliminated. Therefore, loss of light can be suppressed as compared with a case of applying light from the LED light source 10 (20) to the specimen 90 through an air layer. Further, the LED light source 10 (20) can be arranged to be further separated from the specimen 90 by the thickness of the cover portion 407 in addition to the thickness of the sealing portion 406, whereby the photoacoustic imager can deliver the light from the LED light source 10 (20) to a shallower portion of the specimen 90 provided immediately under the detection portion 3.

According to the fourth embodiment, as hereinabove described, the sealing portion 406 is charged into the cover portion 407 in the state where the detection portion 3 and the LED light source 10 (20) are arranged in the cover portion 407, thereby covering the space between the detection portion 3 and the LED light source 10 (20) and the cover portion 407 with no clearance. Thus, the sealing portion 406 can be charged into the cover portion 407 while arranging the detection portion 3 and the LED light source 10 (20) in the cover portion 407, whereby the sealing portion 406 can easily cover the space between the detection portion 3 and the LED light source 10 (20) and the cover portion 407 with no clearance.

Although the present invention has been described and illustrated in detail, it is clearly understood that the same is by way of illustration and example only and is not to be taken by way of limitation, the spirit and scope of the present invention being limited only by the terms of the appended claims.

For example, while the photoacoustic imager includes the acoustic lens in each of the aforementioned first to fourth embodiments, the present invention is not restricted to this. According to the present invention, the photoacoustic imager may alternatively include no acoustic lens.

While the sealing portion is configured to seal the LED light sources in each of the aforementioned first to fourth embodiments, the present invention is not restricted to this. According to the present invention, the sealing portion may not seal the LED light sources, so far as the same seals the surface of the detection portion on the front side in the detection direction.

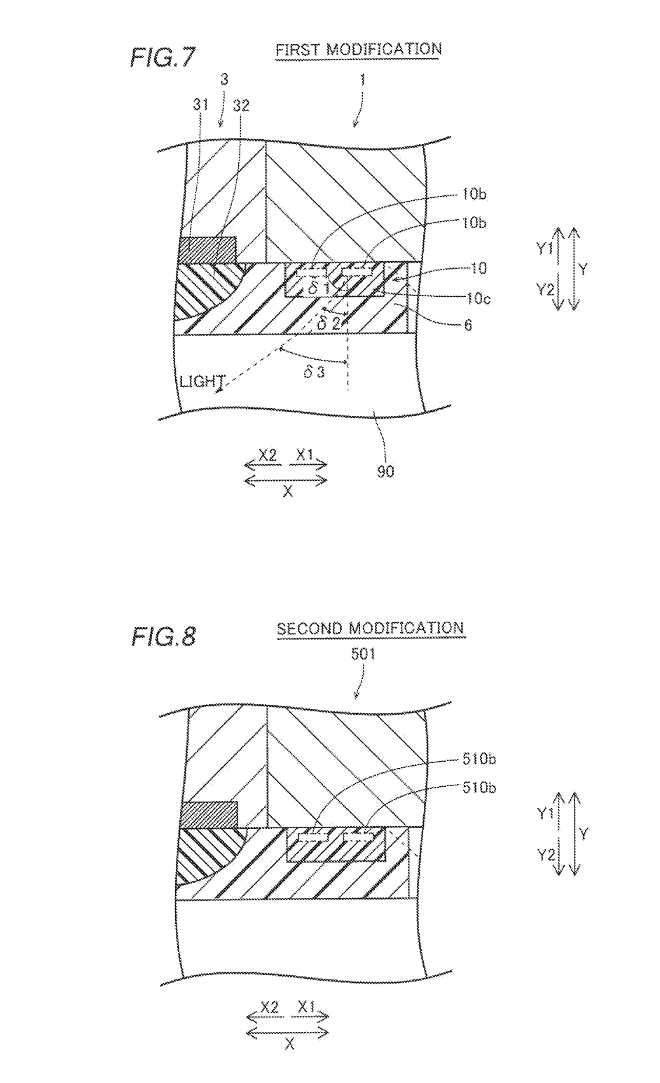

While the refractive indices of the sealing portion and the element sealing portions are identical to each other in each of the aforementioned first to fourth embodiments, the present invention is not restricted to this. According to the present invention, the refractive indices of the sealing portion and the element sealing portions may alternatively be different from each other. For example, a sealing portion 6 may have a smaller refractive index than an element sealing portion 10c, as in a first modification of each of the first to fourth embodiments shown in FIG. 7. In this case, the refractive indices are reduced in order of the element sealing portion 10c, the sealing portion 6 and a specimen 90, and hence a refraction angle δ2 of light from LED elements 10b is larger than an incidence angle δ1 on the boundary surface between the element sealing portion 10c and the sealing portion 6. Further, a refraction angle δ3 of light from the LED elements 10b is larger than an incidence angle δ2 on the boundary surface between the sealing portion 6 and the specimen 90.

While the LED elements are employed as light-emitting semiconductor elements in each of the aforementioned first to fourth embodiments, the present invention is not restricted to this. According to the present invention, light-emitting semiconductor elements other than the LED elements may alternatively be employed. For example, semiconductor laser elements or organic light-emitting diode elements may be employed as light-emitting semiconductor elements 510b, as in a second modification of each of the first to fourth embodiments shown in FIG. 8. In the case of employing semiconductor laser elements, the photoacoustic imager can apply light relatively higher in directivity as compared with light-emitting diode elements to a specimen, whereby the same can reliably apply most part of the light from the semiconductor laser elements to the specimen. In the case of employing organic light-emitting diode elements, on the other hand, a light-emitting semiconductor element light source portion 501 provided with the light-emitting semiconductor elements 501b can be easily miniaturized due to the employment of the organic light-emitting diode elements easily reducible in thickness.

While the sealing portion has the larger refractive index than the cover portion in the aforementioned fourth embodiment, the present invention is not restricted to this. According to the present invention, the sealing portion and the cover portion may alternatively have equal refractive indices. Further alternatively, the sealing portion may have a smaller refractive index than the cover portion.

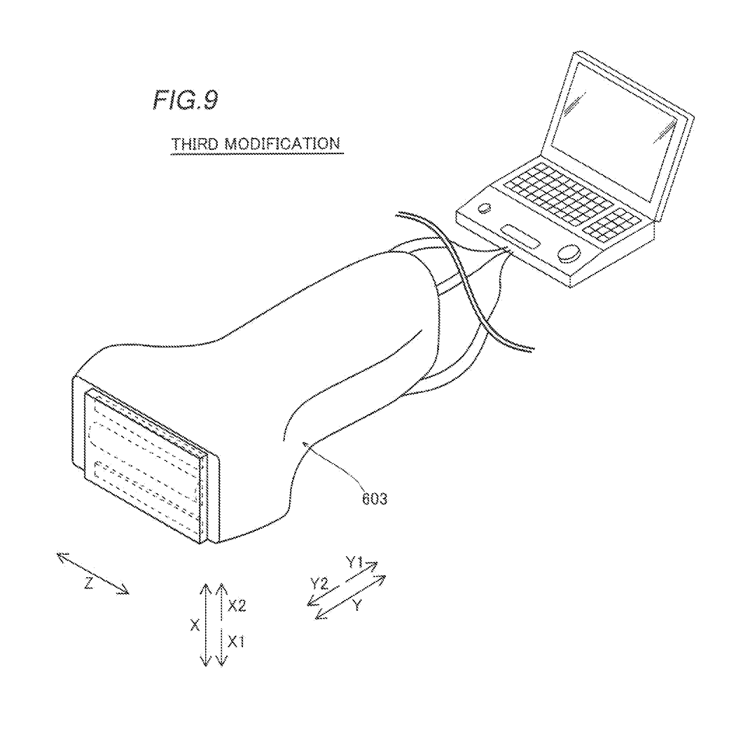

While the detection portion and the light-emitting semiconductor element light source portions are separately provided as light-emitting semiconductor elements in each of the aforementioned first to fourth embodiments, the present invention is not restricted to this. According to the present invention, a detection portion 603 and light-emitting semiconductor element light source portions may alternatively be integrally provided, as in a third modification of each of the first to fourth embodiments shown in FIG. 9.

What is claimed is:

1. A photoacoustic imager comprising:
a light-emitting semiconductor element light source portion including a light-emitting semiconductor element light source outputting light to be applied to a specimen;
a detection portion arranged in proximity to the light-emitting semiconductor element light source portion for detecting an acoustic wave generated by a detection object in the specimen absorbing the light applied to the specimen by the light-emitting semiconductor element light source; and
a sealing portion configured to propagate the acoustic wave generated by the detection object to the detection portion by sealing a surface of the detection portion on a front side in a detection direction where the specimen is arranged with respect to the detection portion and arranged on the front side in the detection direction where the specimen is arranged with respect to the detection portion.

2. The photoacoustic imager according to claim 1, wherein
the sealing portion includes a contact surface coming into contact with the specimen arranged on the front side in the detection direction, and is configured to seal not only the surface of the detection portion but also the light-emitting semiconductor element light source in a state in contact with an emitting surface for light emitted from the light-emitting semiconductor element light source of the light-emitting semiconductor element light source portion on the front side in the detection direction and to transmit the light from the light-emitting semiconductor element light source.

3. The photoacoustic imager according to claim 2, wherein
a pair of the light-emitting semiconductor element light source portions are provided to hold the detection portion therebetween and so configured that an intersection of light emitted from the pair of light-emitting semiconductor element light source portions on the side of the detection portion is positioned on the front side of the detection portion in the detection direction, and
the distance from the light-emitting semiconductor element light source to the contact surface on the front side in the detection direction is larger than the distance from the light-emitting semiconductor element light source to the intersection on the front side in the detection direction.

4. The photoacoustic imager according to claim 2, wherein
the distance from the light-emitting semiconductor element light source to the contact surface on the front side in the detection direction is larger than the thickness of the light-emitting semiconductor element light source.

5. The photoacoustic imager according to claim 1, wherein
the sealing portion covers the detection portion and the light-emitting semiconductor element light source with no clearance.

6. The photoacoustic imager according to claim 1, wherein
the sealing portion includes a curved surface portion arranged on an end on a front side in an emission direction of the light-emitting semiconductor element light source for sealing the detection portion and the light-emitting semiconductor element light source, and is configured to converge light toward the front side of the detection portion in the detection direction by reflecting and refracting the light from the light-emitting semiconductor element light source on the curved surface portion.

7. The photoacoustic imager according to claim 6, wherein
the curved surface portion is provided in the form of an arc smoothly connecting a side end surface of the sealing portion and the contact surface of the sealing portion with each other.

8. The photoacoustic imager according to claim 7, wherein
the side end surface is configured to be substantially flush with a side end surface of the light-emitting semiconductor element light source opposite to the side of the detection portion.

9. The photoacoustic imager according to claim 1, further comprising a cover portion including a contact surface coming into contact with the specimen on the front side in the detection direction and provided in the form of a box opened on the side of the detection portion for propagating the acoustic wave and transmitting the light, wherein
the sealing portion is provided to fill up a space between the cover portion and the detection portion and the light-emitting semiconductor element light source.

10. The photoacoustic imager according to claim 9, wherein
the sealing portion is charged into the cover portion in a state where the detection portion and the light-emitting semiconductor element light source are arranged in the cover portion thereby covering the space between the detection portion and the light-emitting semiconductor element light source and the cover portion with no clearance.

11. The photoacoustic imager according to claim 9, wherein
the light-emitting semiconductor element light source has a plurality of light-emitting semiconductor elements and an element sealing portion constituting the light-emitting semiconductor element light source along with the plurality of light-emitting semiconductor elements by sealing the light-emitting semiconductor elements, and
the sealing portion has a refractive index larger than the refractive index of the cover portion and not more than the refractive index of the element sealing portion.

12. The photoacoustic imager according to claim 1, wherein
the light-emitting semiconductor element light source has a plurality of light-emitting semiconductor elements and an element sealing portion constituting the light-emitting semiconductor element light source along with the plurality of light-emitting semiconductor elements by sealing the light-emitting semiconductor elements, and
the sealing portion has a refractive index larger than the refractive index of the specimen and not more than the refractive index of the element sealing portion.

13. The photoacoustic imager according to claim 1, wherein
the detection portion includes an ultrasonic vibrator detecting the acoustic wave as an ultrasonic wave and an acoustic lens arranged on the front side of the ultrasonic vibrator in the detection direction in a state sealed by the sealing portion for converging the acoustic wave from the detection object on the ultrasonic vibrator, and
the sealing portion has larger transmittance than the acoustic lens and equivalent ultrasonic wave propagation loss to the acoustic lens.

14. The photoacoustic imager according to claim 13, wherein
the acoustic lens is provided in a rounded convex shape protruding on the front side in the detection direction.

15. The photoacoustic imager according to claim 13, wherein
the sealing portion is arranged between the acoustic lens and the light-emitting semiconductor element light source with no clearance.

16. The photoacoustic imager according to claim 13, wherein
the sealing portion includes a contact surface coming into contact with the specimen arranged on the front side in the detection direction, and
the contact surface is arranged substantially parallelly with a light-emitting surface of the light-emitting semiconductor element light source on the front side in the detection direction and a surface of the ultrasonic vibrator on the front side in the detection direction.

17. The photoacoustic imager according to claim 1, wherein
the detection portion and the light-emitting semiconductor element light source are formed to extend in the same direction orthogonal to the front side in the detection direction.

18. The photoacoustic imager according to claim 1, wherein
the light-emitting semiconductor element light source portion includes a light-emitting diode element as a light-emitting semiconductor element.

19. The photoacoustic imager according to claim 1, wherein
the light-emitting semiconductor element light source portion includes a semiconductor laser element as a light-emitting semiconductor element.

20. The photoacoustic imager according to claim 1, wherein
the light-emitting semiconductor element light source portion includes an organic light-emitting diode element as a light-emitting semiconductor element.
